(19) United States
(12) Patent Application Publication
(10) Pub. No.: US 2024/0259653 A1
(43) Pub. Date: Aug. 1, 2024

(54) SIGNAL PROCESSING SYSTEM AND SIGNAL PROCESSING METHOD

(71) Applicant: SONY GROUP CORPORATION, TOKYO (JP)

(72) Inventors: MASAHITO YAMANE, TOKYO (JP); TAKUYA MIYAZAKI, TOKYO (JP); YUKI SUGIE, TOKYO (JP)

(21) Appl. No.: 18/560,719

(22) PCT Filed: Feb. 9, 2022
(86) PCT No.: PCT/JP2022/004996

§ 371 Date: Nov. 14, 2023

(30) Foreign Application Priority Data
Jun. 3, 2021 (JP) 2021-093711

(51) Int. Cl. H04N 21/8547; G16H 30/00; H04N 7/18; H04N 21/24

(52) U.S. Cl. CPC H04N 21/8547; H04N 7/18; H04N 21/24; G16H 30/00

(57) ABSTRACT

The present disclosure relates to a signal processing system and a signal processing method that enable realization of display optimization in a real-time service. A transmission device transmits an image signal from an imaging device, a processing device provided on a cloud performs signal processing on the transmitted image signal, and a reception device receives the image signal subjected to the signal processing. An integration device is connected to a network together with the transmission device, the processing device, and the reception device which are time-synchronized, sets a signal delay amount from the transmission device to the reception device in accordance with a signal processing content of the processing device, and transmits the signal delay amount to the reception device. The present disclosure can be applied to a network system for medical use and a network system for use in a broadcasting station.

### TECHNICAL FIELD

The present disclosure relates to a signal processing system and a signal processing method, and more particularly, to a signal processing system and a signal processing method for realizing display optimization in a real-time service.

### BACKGROUND ART

It has been proposed that a plurality of servers on a cloud executes various types of software signal processing on image signals in real-time services requiring real-time properties, such as medical services and broadcast services with development of communication technologies and an improvement in performance of general-purpose processors.

In the real-time services, extremely low latency close to an on-premises environment is required, and thus, there is a demand for establishment of various low-latency technologies and technologies that do not make users feel latency.

Patent Document 1 discloses a remote control method in which a control device having a monitor for viewing an image, provided by a camera arranged at a remote location, provides an emulated image for display prior to execution of a command at the camera so as to prevent a user from feeling a delay.

### CITATION LIST

### Patent Document

Patent Document 1: Japanese Patent Application Laid-Open No. 2019-186934

### SUMMARY OF THE INVENTION

### Problems to be Solved by the Invention

There is a case where a processing time in each of the servers on the cloud is different for each of the servers and each of applications. That is, there is a case where a delay time is different for each type of software signal processing. Therefore, for example, in a case where a plurality of processing results is integrated and displayed in one image in order of completion of each type of processing, acquisition time is different for each image region, and thus, there is a possibility that display is inappropriate.

Therefore, there is a demand for a system capable of realizing optimization of display in accordance with each processing time when a plurality of different types of signal processing is executed in real time.

The present disclosure has been made in view of such a situation, and realizes optimization of display in a real-time service.

### Solutions to Problems

A signal processing system of the present disclosure is a signal processing system including: a transmission device that transmits an image signal from an imaging device; a processing device that is provided on a cloud and performs signal processing on the transmitted image signal; a reception device that receives the image signal subjected to the signal processing; and an integration device that is connected to a network together with the transmission device, the processing device, and the reception device which are time-synchronized, sets a signal delay amount from the transmission device to the reception device in accordance with a signal processing content of the processing device, and transmits the signal delay amount to the reception device.

A signal processing method of the present disclosure is a signal processing method including setting, by an integration device of a signal processing system, a signal delay amount from a transmission device to a reception device in accordance with a signal processing content of a processing device and transmitting the signal delay amount to the reception device, the signal processing system including: the transmission device that transmits an image signal from an imaging device; the processing device that is provided on a cloud and performs signal processing on the transmitted image signal; the reception device that receives the image signal subjected to the signal processing; and the integration device that is connected to a network together with the transmission device, the processing device, and the reception device which are time-synchronized.

In the present disclosure, the signal delay amount from the transmission device to the reception device is set in accordance with the signal processing content of the processing device, and is transmitted to the reception device.

### BRIEF DESCRIPTION OF DRAWINGS

FIG. 1 is a block diagram illustrating a configuration example of a signal processing system to which the technology according to the present disclosure can be applied.

FIG. 2 is a block diagram illustrating a hardware configuration example of a computer.

FIG. 3 is a diagram illustrating a functional configuration example of an integration device.

FIG. 4 is a diagram for describing setting of a signal delay amount.

FIG. 5 is a diagram for describing a flow from acquisition to output of an image signal.

FIG. 6 is a diagram illustrating an example of a divided image.

FIG. 7 is a diagram illustrating an example of performing signal processing on a plurality of image signals.

FIG. 8 is a view for describing latency-prioritized synchronization.

FIG. 9 is a view for describing time-prioritized synchronization.

### MODE FOR CARRYING OUT THE INVENTION

A mode for carrying out the present disclosure (hereinafter, referred to as an embodiment) will be described below. Note that, the description will be given in the following order.
1. Background Art and Problems Thereof
2. Configuration of Signal Processing System
3. Flow from Acquisition to Output of Image Signal
4. Signal Processing on Cloud and Synchronization

### 1. Background Art and Problems Thereof

It has been proposed that a plurality of servers on a cloud executes various types of software signal processing on image signals in real-time services requiring real-time properties, such as medical services and broadcast services with development of communication technologies and an improvement in performance of general-purpose processors.

In the real-time services, extremely low latency close to an on-premises environment is required, and thus, there is a demand for establishment of various low-latency technologies and technologies that do not make users feel latency.

There is a case where a processing time in each of the servers on the cloud is different for each of the servers and each of applications. That is, there is a case where a delay time is different for each type of software signal processing. For example, in a medical service, a processing time is greatly different between artificial intelligence (AI) diagnosis application processing on an endoscopic image and up-conversion processing on an endoscopic image. Therefore, for example, in a case where these processing results are integrated and displayed in one image in order of completion of each type of processing, acquisition time is different for each image region, and thus, there is a possibility that display is inappropriate due to a frame drop.

Therefore, there is a demand for a system capable of realizing optimization of display in accordance with each processing time when a plurality of different types of signal processing is executed in real time.

Hereinafter, a configuration of a signal processing system that realizes optimization of display in a real-time service will be described.

### <2. Configuration of Signal Processing System>

### (Configuration Example of Entire Signal Processing System)

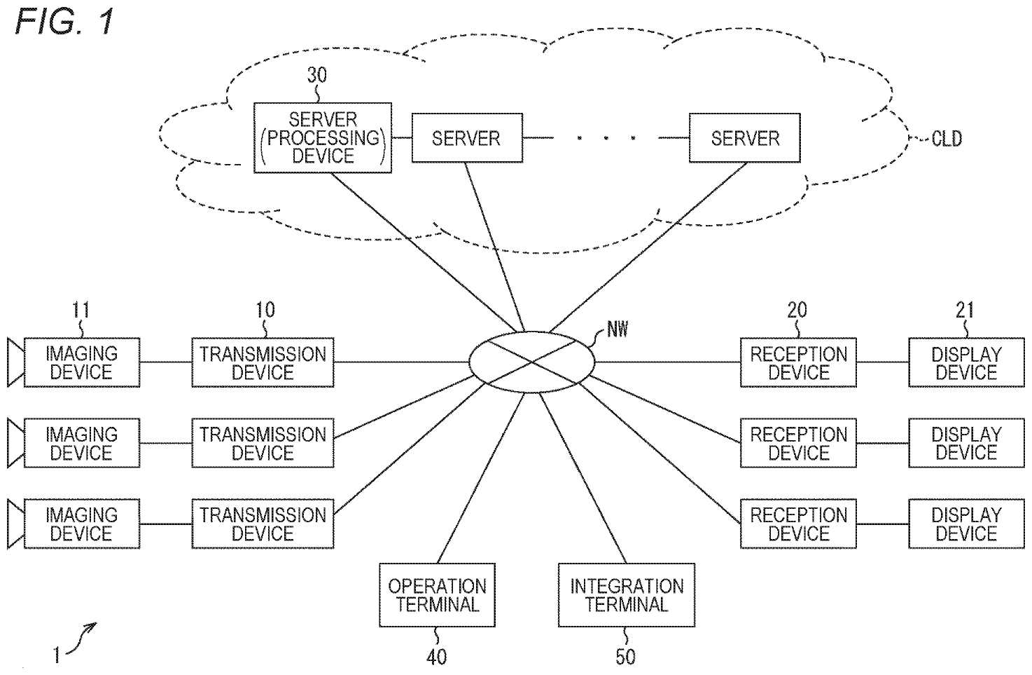

FIG. 1 is a block diagram illustrating a configuration example of a signal processing system to which the technology according to the present disclosure can be applied.

A signal processing system 1 in FIG. 1 may be configured as a network system for medical use that realizes provision of a medical service, or may be configured as a network system for use in a broadcasting station that realizes provision of a broadcasting service.

The signal processing system 1 includes transmission devices 10, imaging devices 11, reception devices 20, display devices 21, servers 30, an operation terminal 40, and an integration device 50.

In the signal processing system 1, a plurality of the imaging devices 11 is provided, and the transmission devices 10 as many as the imaging devices 11 are also provided. Similarly, a plurality of the display devices 21 is provided, and the reception devices 20 as many as the display devices 21 are also provided.

In the signal processing system 1, the transmission devices 10, the reception devices 20, the server 30, the operation terminal 40, and the integration device 50 are connected to a network NW forming a so-called Internet Protocol (IP) network.

The imaging devices 11 are configured as electronic devices each having a function of capturing a moving image. In a case where the signal processing system 1 is configured as the network system for medical use, the imaging devices 11 are configured as medical imaging devices (medical devices) such as an endoscope and a surgical field camera. In a case where the signal processing system 1 is configured as the network system for use in a broadcasting station, the imaging device 11 is configured as a video camera for broadcasting or the like.

The display devices 21 are configured as monitors each displaying the image (moving image) captured by the imaging device 11 in real time.

The imaging devices 11 are connected to the network NW via the transmission devices 10, respectively. Furthermore, the display devices 21 are connected to the network NW via the reception devices 20, respectively. The imaging devices 11 and the display devices 21 have interfaces such as a serial digital interface (SDI), a high-definition multimedia interface (HDMI) (registered trademark), and a display port.

The transmission devices 10 and the reception devices 20 are configured as so-called IP converters. The transmission device 10 converts the image (an image signal) from the imaging device 11 into an IP signal and transmits the IP signal to the network NW. Furthermore, the reception device 20 converts the IP signal received from the network NW into an image signal and outputs the image signal to the display device 21.

Although the transmission device 10 and the imaging device 11 are separately configured in the example of FIG. 1, these may be integrally configured. Furthermore, the reception device 20 and the display device 21 are also separately configured, but these may be integrally configured.

The plurality of the servers 30 is configured as processing devices each performing image processing on an image signal, and are provided on a cloud CLD. The server 30 acquires the image signal transmitted from the transmission device 10 via the network NW and performs signal processing by software.

For example, in a case where the signal processing system 1 is configured as the network system for medical use, the server 30 superimposes and combines a guide on an endoscopic image and overlays a fluorescence image on a 4K full-color image. In a case where the signal processing system 1 is configured as the network system for used in a broadcast station, the server 30 performs picture in picture (PinP) processing on a plurality of images, superimposes a logo or a telop, or adds an effect.

The server 30 transmits the image signal subjected to the image processing to the reception device 20 via the network NW. Routing between the transmission device 10 and the reception device 20 is performed on the basis of control of the integration device 50.

The plurality of servers 30 provided on the cloud CLD can receive the image signals from the plurality of transmission devices 10, perform various types of image processing for each of the servers 30, and transmit the processed image signals to the plurality of reception devices 20.

The operation terminal 40 is configured as a personal computer (PC), a tablet terminal, a smartphone, or the like operated by a user. For example, the operation terminal 40 receives selection of the reception device 20 as an output destination of the image signal transmitted from the transmission device 10 on the basis of an operation of the user.

The integration device 50 controls input and output of an image signal to and from a device connected to the network NW. Specifically, the integration device 50 controls high-speed transfer of an image signal between the transmission device 10 and the reception device 20 arranged on the network NW. That is, the integration device 50 sets (performs routing) an output destination of the image signal transmitted from the transmission device 10 and the reception device 20 by controlling an IP switch (not illustrated) on the basis of the user operation on the operation terminal 40.

Moreover, the integration device 50 sets a signal processing content of each of the servers 30 on the cloud CLD on the basis of the user operation on the operation terminal 40. Each of the servers 30 performs image processing on the image signal transmitted from the transmission device 10 on the basis of the signal processing content set by the integration device 50.

Note that the imaging device 11 (the transmission device 10), the display device 21 (the reception device 20), and the server 30 are time-synchronized in the signal processing system 1 in order to realize transmission of an image signal with low latency. Here, for example, clock synchronization using a precision time protocol (PTP) between an edge and a cloud and time synchronization using a grandmaster clock (GMC) are executed.

### (Hardware Configuration Example of Computer)

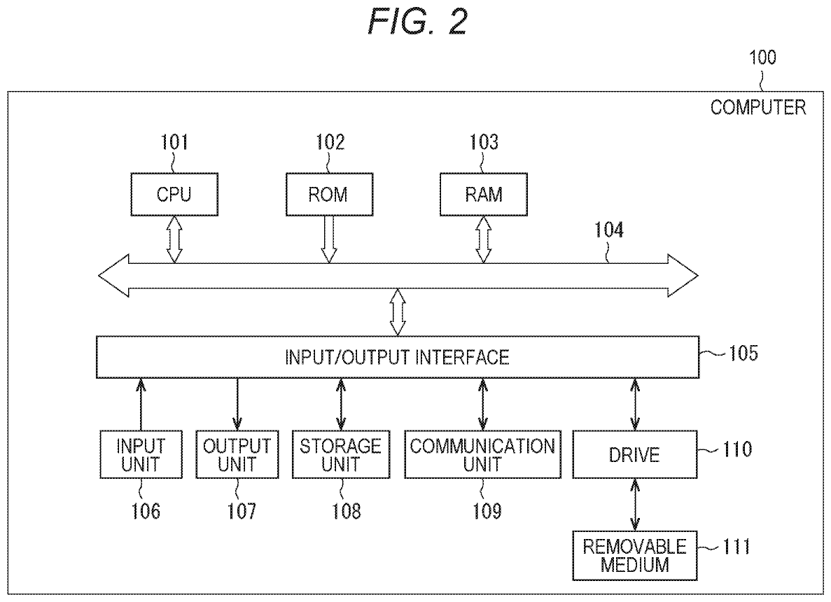

FIG. 2 is a block diagram illustrating a hardware configuration example of a computer forming each device of the signal processing system 1 described above.

The servers 30, the operation terminal 40, and the integration device 50 constituting the signal processing system 1 are realized by computers 100 having configurations illustrated in FIG. 2.

A central processing unit (CPU) 101, a read only memory (ROM) 102, and a random access memory (RAM) 103 are mutually connected by a bus 104.

The bus 104 is further connected with an input/output interface 105. An input unit 106 including a keyboard, a mouse, a button, a touch panel, and the like, and an output unit 107 including a display, a speaker, and the like are connected to the input/output interface 105. Furthermore, the input/output interface 105 is connected with a storage unit 108 including a hard disk, a non-volatile memory, and the like, a communication unit 109 including a network interface and the like, and a drive 110 that drives a removable medium 111.

In the computer 100 configured as described above, for example, the CPU 101 loads a program stored in the storage unit 108 into the RAM 103 via the input/output interface 105 and the bus 104 and executes the program, thereby executing various processes.

The program to be executed by the CPU 101 is provided, for example, by being recorded on the removable medium 111 or via a wired or wireless transmission medium such as a local area network, the Internet, or digital broadcasting, and is installed in the storage unit 108.

In a case where the computers 100 are configured as the servers 30, a graphics processing unit (GPU) or a field programmable gate array (FPGA) may be further provided in addition to the configurations in FIG. 2. Therefore, the respective servers 30 on the cloud CLD can execute real-time signal processing in parallel with low latency.

### (Functional Configuration Example of Integration Device)

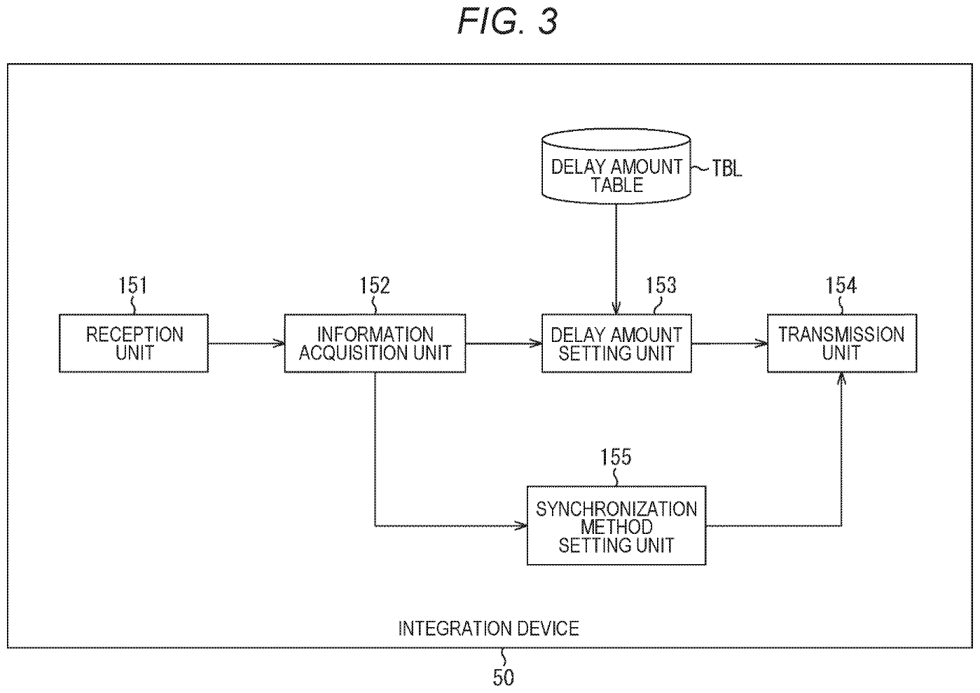

Next, a functional configuration example of the integration device 50 will be described with reference to FIG. 3.

The integration device 50 in FIG. 3 includes a reception unit 151, an information acquisition unit 152, a delay amount setting unit 153, a transmission unit 154, a synchronization method setting unit 155, and a delay amount table TBL. The information acquisition unit 152, the delay amount setting unit 153, and the synchronization method setting unit 155 are functional blocks realized as the CPU 101 in FIG. 2 executes predetermined programs.

The reception unit 151 corresponds to the communication unit 109 in FIG. 2, and receives various types of information from the transmission device 10, the reception device 20, the server 30, and the operation terminal 40 via the network NW.

The information acquisition unit 152 acquires various types of information received by the reception unit 151 and supplies the information to the delay amount setting unit 153 and the synchronization method setting unit 155. The information acquired by the information acquisition unit 152 includes, for example, the signal processing content set for each of the servers 30 on the cloud CLD, operation information of the user who operates the operation terminal 40, and the like.

The delay amount setting unit 153 sets a time difference (hereinafter, referred to as a signal delay amount) from when transmission of an image signal is started by the transmission device 10 to when the image signal is received by the reception device 20 and display of the image is started by the display device 21 on the basis of the information from the information acquisition unit 152. Specifically, the delay amount setting unit 153 sets a signal delay amount from the transmission device 10 to the reception device 20 in accordance with the signal processing content of each of the servers 30.

The delay amount table TBL corresponds to the storage unit 108 of FIG. 2, and holds a processing time (worst case execution time) of signal processing by each of the servers 30 for each of the servers 30 on the cloud CLD.

The delay amount setting unit 153 can also set a signal delay amount from the transmission device 10 to the reception device 20 on the basis of the processing time of each of the servers 30 held in the delay amount table TBL.

The signal delay amount set by the delay amount setting unit 153 is supplied to the transmission unit 154.

The transmission unit 154 corresponds to the communication unit 109 in FIG. 2 similarly to the reception unit 151, and transmits the signal delay amount from the delay amount setting unit 153 to the reception device 20.

The synchronization method setting unit 155 sets a synchronization method of signal processing executed in the servers 30 on the cloud CLD on the basis of the information from the information acquisition unit 152. Specifically, the synchronization method of signal processing is set on the basis of a user operation on the operation terminal 40. Information indicating the set synchronization method is supplied to the transmission unit 154 and transmitted to the server 30 that executes the corresponding signal processing.

### (Setting of Signal Delay Amount)

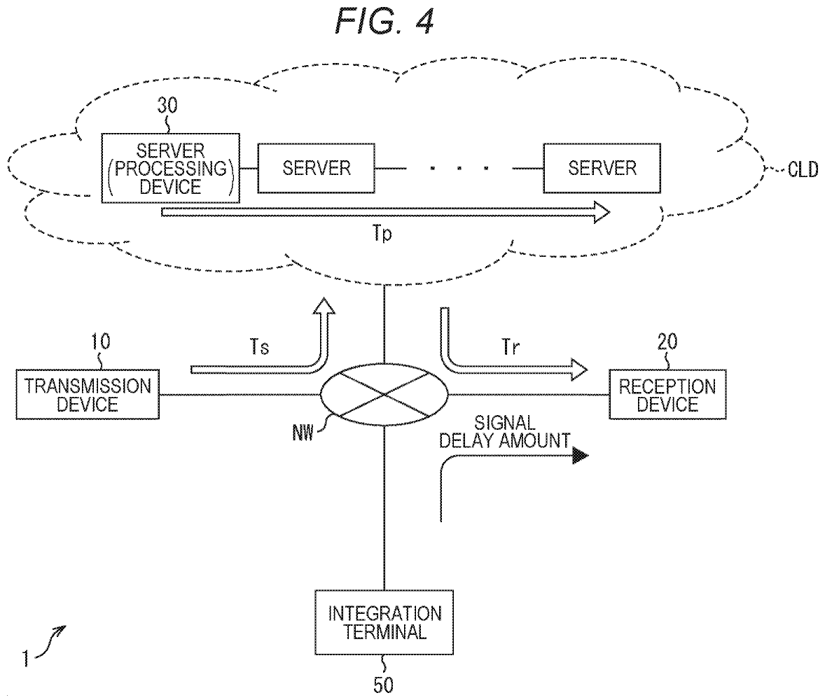

Here, setting of the signal delay amount by the delay amount setting unit 153 will be described with reference to FIG. 4.

FIG. 4 illustrates a transmission time Ts until an image signal transmitted from the transmission device 10 is transmitted to the server 30, a processing time Tp in each of the servers 30 on the cloud CLD, and a transmission time Tr until the image signal transmitted from the server 30 is transmitted to the reception device 20.

On the cloud CLD, each of the servers 30 starts signal processing as soon as receiving a frame (an image signal) from a preceding stage, and sends a frame as a processing result to a subsequent stage. Therefore, the processing time Tp is assumed to include a transmission time between the servers 30.

The delay amount setting unit 153 (the integration device 50) sets a signal delay amount on the basis of the transmission time Ts, the processing time Tp, and the transmission time Tr, and transmits the signal delay amount to the reception device 20. Specifically, the delay amount setting unit 153 sets the signal delay amount on the basis of a time obtained by adding the maximum times of the transmission time Ts, the processing time Tp, and the transmission time Tr. The delay amount setting unit 153 transmits the set signal delay amount to the reception device 20 in addition to time information (imaging time by the imaging device 11) included in the image signal received by the reception device 20.

The reception device 20 outputs the received image signal at a timing determined by the time information (imaging time) included in the image signal and the signal delay amount added to the time information.

It is sufficient to dynamically calculate each of the transmission times Ts and Tr on the basis of an existing image transmission technology. Furthermore, the processing time Tp on the cloud CLD may be obtained by a dynamic method or may be obtained by a static method.

The dynamic method is a method in which the integration device 50 controls the transmission device 10, the reception device 20, and the server 30 to execute image signal transmission and signal processing, and actually measures and aggregates times required for the transmission and signal processing.

The static method is a method in which the integration device 50 refers to the delay amount table TBL (FIG. 3) and calculates a processing time in accordance with signal processing executed in each of the servers 30.

<3. Flow from Acquisition to Output of Image Signal>

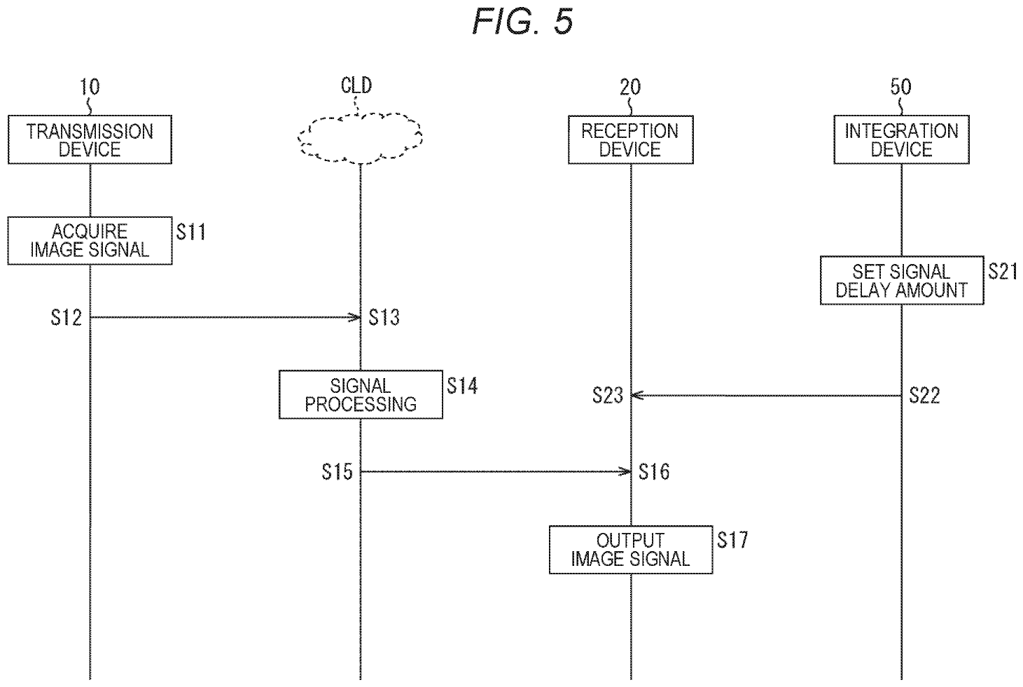

FIG. 5 is a diagram for describing a flow from acquisition to output of an image signal in the signal processing system 1. Here, an example in which an image captured by one imaging device 11 is displayed on one display device 21 in real time will be described.

When an image (an image signal) captured by the imaging device 11 is acquired in a unit of a frame in step S11, the transmission device 10 sends the acquired image signal in a unit of a frame to the cloud CLD (the server 30) in step S12.

When the cloud CLD (the plurality of servers 30) receives the image signal in a unit of a frame from the transmission device 10 in step S13, the cloud CLD executes signal processing by software on the image signal in a unit of a frame in step S14. In step S15, the cloud CLD transmits the image signal subjected to the signal processing to the reception device 20.

When the image signal is received from the cloud CLD in step S16, the reception device 20 outputs the received image signal to the display device 21 in step S17.

On the other hand, the integration device 50 sets a signal delay amount in step S21, and transmits the set signal delay amount to the reception device 20 in step S22. In step S23, the reception device 20 receives the signal delay amount from the integration device 50.

Therefore, in step S17, the reception device 20 outputs the image signal received from the cloud CLD to the display device 21 at a timing based on the signal delay amount from the integration device 50.

According to the above processing, in the signal processing system 1 in which the transmission device 10, the reception device 20, and the server 30 (the cloud CLD) are time-synchronized, the display device 21 can display the image at the timing based on the signal delay amount set by the integration device 50.

Therefore, even when processing results are integrated into one image and displayed in a case where the processing time in each of the servers on the cloud is different for each of the servers and for each of applications, it is possible to realize display optimization in accordance with the processing time for each of the servers and for each of the applications without causing a frame drop.

Note that the signal delay amount set by the integration device 50 is transmitted to the reception device 20 in the above description, but may be transmitted to each of the servers 30 on the cloud CLD. Therefore, each of the servers 30 can execute the signal processing at the timing based on the signal delay amount set by the integration device 50, and can contribute to the realization of display optimization in accordance with the processing time for each of the servers and each of the applications.

### <4. Signal Processing on Cloud and Synchronization>

There is known a technology in which an image is divided in the horizontal direction and allocated to a plurality of processors, and the respective processors perform time-division processing on an allocated region in a vertical direction and sequentially process the respective regions divided in the vertical direction with the largest overhead set to a head region, thereby displaying the image at high speed.

In order to achieve low latency in the transmission and signal processing of the image signal on the cloud CLD in the signal processing system 1, each of the servers 30 executes the transmission and signal processing in a unit of a divided image obtained by dividing each frame of the image signal.

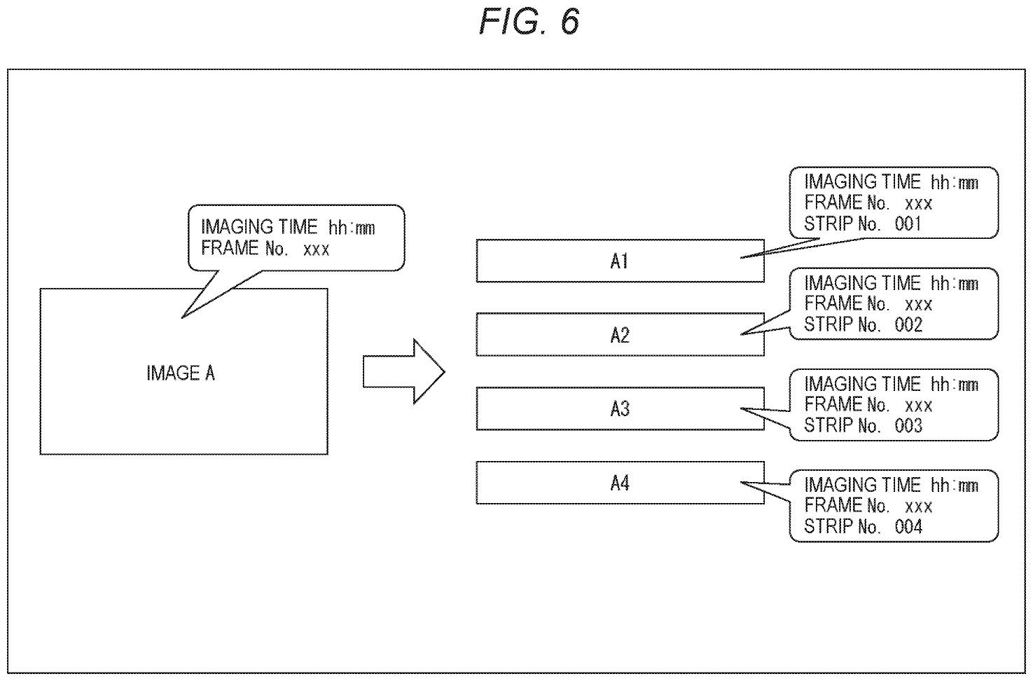

Specifically, as illustrated in FIG. 6, a predetermined frame of an image A is divided into four in the horizontal direction. Images corresponding to four regions A1, A2, A3, and A4 obtained by dividing the image A are referred to as strip images, respectively. Imaging time (time information) and a frame number are added to the image A as metadata. Imaging time, a frame number, and a strip number (divided image number) are added as metadata to each of the strip images A1, A2, A3, and A4 on the basis of the metadata of the image A. In the example of FIG. 6, strip number 001 is added to the strip image A1, and strip number 002 is added to the strip image A2. Similarly, strip number 003 is added to the strip image A3, and strip number 004 is added to the strip image A4.

On the cloud CLD, each of the servers 30 starts signal processing as soon as receiving a strip image to which a strip number is added, and sends the strip image as a processing result to a subsequent stage.

In such a configuration, when the signal processing is performed during transmission of image signals between the plurality of servers 30, a time difference may be generated in units of strip images for a plurality of image signals input to a certain server 30. In this case, synchronization needs to be performed in a unit if a frame, for example, when image synthesis processing such as PinP processing is executed.

Therefore, in a case where the signal processing is performed on the plurality of image signals, the server 30 executes the synchronization in a unit of a frame on the basis of a result of the signal processing in a unit of a divided image. At this time, the server 30 executes the synchronization in a unit of a frame by using metadata (an imaging time, a frame number, and a divided image number) added to the divided image.

As a synchronization method in a unit of a frame, the server 30 can execute either latency-prioritized synchronization in which a first image signal is not delayed but is synchronized with a second image signal preceding by the predetermined number of frames, or time-prioritized synchronization in which the first image signal is delayed and synchronized in accordance with the second image signal.

Then, which one of the latency-prioritized synchronization and the time-prioritized synchronization is executed by each of the servers 30 on the cloud CLD is set by the synchronization method setting unit 155 of the integration device 50.

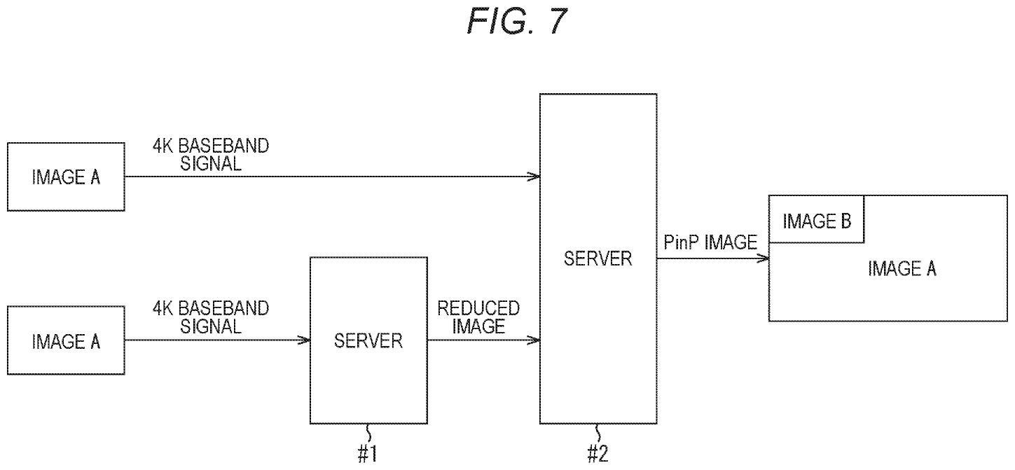

FIG. 7 is a diagram illustrating an example of performing signal processing on a plurality of image signals.

In the example of FIG. 7, two images A and B are displayed as one image (a PinP image) by PinP processing. Specifically, a server #1 performs reduction processing on the image B out of the two images A and B input as a 4K baseband signal, and outputs the image B to a server #2. The server #2 executes the PinP processing on the image A input as the 4K baseband signal and the image B input as the reduced image to output the PinP image in which the reduced image (the image B) is superimposed on the image A.

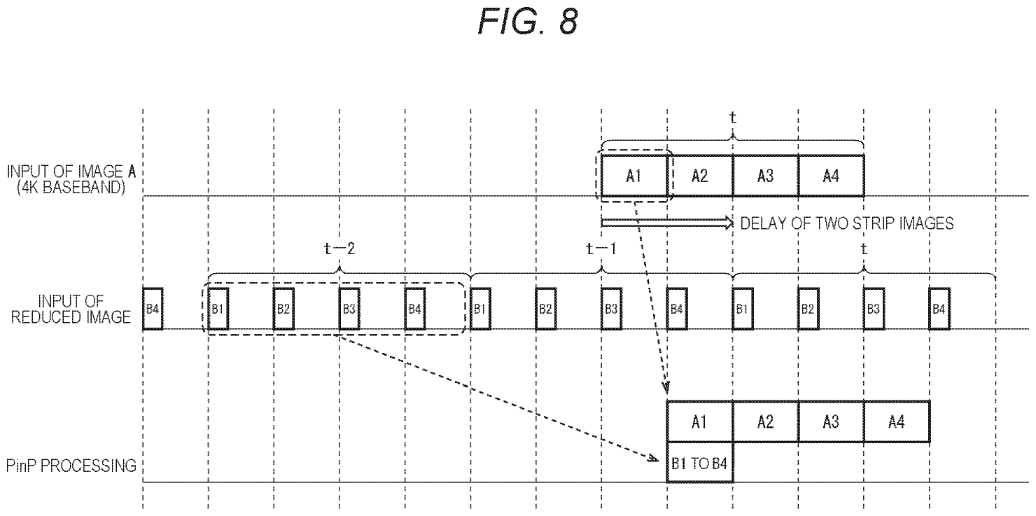

The latency-prioritized synchronization in the configuration of FIG. 7 will be described with reference to FIG. 8.

The upper part of FIG. 8 illustrates a frame (hereinafter, referred to as a frame A(t) and the like) of the image A input to the server #2 at time t for each of the strip images A1, A2, A3, and A4. The middle part of FIG. 8 illustrates frames (hereinafter, referred to as frames B(t−2), B(t−1), B(t), and the like) at time t−2, time t−1, and time t of the reduced image (the image B) input to the server #2 for each of the strip images B1, B2, B3, and B4.

Since the image B is subjected to the reduction processing in the server #1, the reduced image is input to the server #2 with a delay of two strip images from the image A in the example of FIG. 8.

Here, when the server #2 executes the PinP processing on the image A and the reduced image, the PinP processing is executed by performing synchronization with the frame B(t−2) preceding by two frames without delaying the frame A(t) on the basis of the metadata of the strip images of the images A and B.

Therefore, the PinP image can be output fastest although time is not synchronized between the image A and the reduced image (the image B).

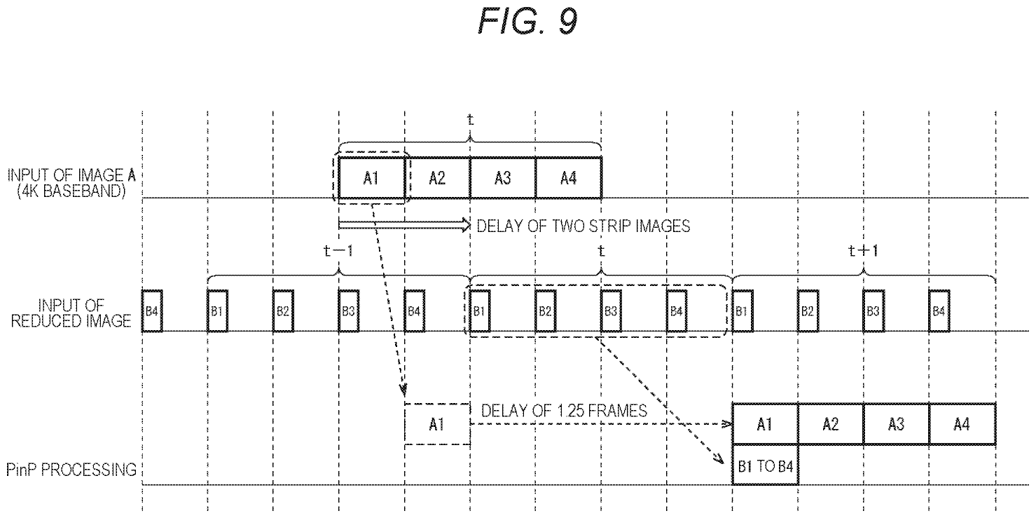

The time-prioritized synchronization in the configuration of FIG. 7 will be described with reference to FIG. 9.

The upper part of FIG. 9 illustrates the frame A(t) of the image A input to the server #2 at time t for each of the strip images A1, A2, A3, and A4. The middle part of FIG. 9 illustrates the frames B(t−1), B(t), and B(t+1) at time t−1, time t, and time t+1 of the reduced image (the image B) input to the server #2 for each of the strip images B1, B2, B3, and B4.

Since the image B is subjected to the reduction processing in the server #1, the reduced image is input to the server #2 with a delay of two strip images from the image A in the example of FIG. 9.

Here, when the server #2 executes the PinP processing on the image A and the reduced image, the PinP processing is executed by delaying the frame A(t) by 1.25 frames and synchronized in accordance with the frame B(t) on the basis of the metadata of the strip images of the images A and B. Therefore, it is possible to output the PinP image obtained by time synchronization between the image A and the reduced image (the image B).

In a case where the signal processing system 1 is configured as the network system for used in a broadcast station, it is possible to realize broadcast that does not give a viewer a sense of discomfort by selecting the time-prioritized synchronization when a video to be viewed by the viewer is to be output. On the other hand, more rapid checking can be realized by selecting the latency-prioritized synchronization when a video for checking in a broadcast station is to be output.

Furthermore, in a case where the signal processing system 1 is configured as the network system for medical use, either the latency-prioritized synchronization or the time-prioritized synchronization may be set on the basis of a type of the imaging device 11 as a medical device or a type of a medical application that executes signal processing. The time-prioritized synchronization is selected, for example, in a case where a guide is superimposed and displayed on an image input as a baseband signal. The latency-prioritized synchronization is selected in a case where an image input as a baseband signal is displayed as a proxy image with reduced resolution.

In the signal processing system 1 described above, GPUDirect remote direct memory access (RDMA), which is one type of GPUDirect (registered trademark) that directly transfers data from another server to a GPU without passing through a CPU, may be used for image signal transmission between the servers 30. Therefore, it is possible to further reduce latency related to the image signal transmission.

Embodiments of the present disclosure are not limited to the above-described embodiment, and various modifications can be made in a range without departing from the gist of the present disclosure.

Furthermore, effects described in the present specification are merely illustrative and not restrictive, and may include other effects.

Moreover, the present disclosure may have the following configurations.

(1)

A signal processing system including:
a transmission device that transmits an image signal from an imaging device;
a processing device that is provided on a cloud and performs signal processing on the transmitted image signal;
a reception device that receives the image signal subjected to the signal processing; and
an integration device that is connected to a network together with the transmission device, the processing device, and the reception device which are time-synchronized, sets a signal delay amount from the transmission device to the reception device in accordance with a signal processing content of the processing device, and transmits the signal delay amount to the reception device.
   (2)

The signal processing system according to (1), in which the reception device outputs the received image signal at a timing based on the signal delay amount.

(3)

The signal processing system according to (2), in which the integration device adds the signal delay amount to time information included in the image signal received by the reception device, and the reception device outputs the received image signal at the timing determined by the time information and the signal delay amount.

(4)

The signal processing system according to (3), in which the time information includes imaging time by the imaging device.

(5)

The signal processing system according to any one of (1) to (4), in which the integration device sets the signal delay amount on the basis of a transmission time from the transmission device to the processing device, a processing time in the processing device, and a transmission time from the processing device to the reception device.

(6)

The signal processing system according to (5), in which the integration device calculates the processing time using a delay amount table regarding the signal processing by the processing device provided on the cloud.

(7)

The signal processing system according to (6), in which
a plurality of the processing devices is provided on the cloud, and
the processing time includes a transmission time between the processing devices.
   (8)

The signal processing system according to any one of (1) to (7), in which the processing device performs the signal processing on the image signal in a unit of a divided image obtained by dividing a frame.

(9)

The signal processing system according to (8), in which in a case where the signal processing is performed on a plurality of the image signals, the processing device executes synchronization in a unit of the frame on the basis of a result of the signal processing performed in the unit of the divided image.

(10)

The signal processing system according to (9), in which the integration device sets, as a method for the synchronization executed in the unit of the frame, either latency-prioritized synchronization in which a first image signal is not delayed but is synchronized with a second image signal preceding by the predetermined number of frames, or time-prioritized synchronization in which the first image signal is delayed and synchronized in accordance with the second image signal.

(11)

The signal processing system according to (10), in which
the imaging device includes a medical device, and
the integration device sets either the latency-prioritized synchronization or the time-prioritized synchronization on the basis of a type of the medical device.
   (12)

The signal processing system according to (10) or (11), in which the processing device performs either the latency-prioritized synchronization or the time-prioritized synchronization using time information, a frame number, and a divided image number included in the image signal.

(13)

A signal processing method including
setting, by an integration device of a signal processing system, a signal delay amount from a transmission device to a reception device in accordance with a signal processing content of a processing device and transmitting the signal delay amount to the reception device, the signal processing system including:
the transmission device that transmits an image signal from an imaging device;
the processing device that is provided on a cloud and performs signal processing on the transmitted image signal;
the reception device that receives the image signal subjected to the signal processing; and
the integration device that is connected to a network together with the transmission device, the processing device, and the reception device which are time-synchronized.

### REFERENCE SIGNS LIST

1 Signal processing system
10 Transmission device
11 Imaging device
20 Reception device
21 Display device
30 Server
40 Operation terminal
50 Integration terminal
151 Reception unit
152 Information acquisition unit
153 Delay amount setting unit
154 Transmission unit
155 Synchronization method setting unit
NW Network
CLD Cloud
TBL Delay amount table

1. A signal processing system comprising:
a transmission device that transmits an image signal from an imaging device;
a processing device that is provided on a cloud and performs signal processing on the transmitted image signal;
a reception device that receives the image signal subjected to the signal processing; and
an integration device that is connected to a network together with the transmission device, the processing device, and the reception device which are time-synchronized, sets a signal delay amount from the transmission device to the reception device in accordance with a signal processing content of the processing device, and transmits the signal delay amount to the reception device.

2. The signal processing system according to claim 1, wherein the reception device outputs the received image signal at a timing based on the signal delay amount.

3. The signal processing system according to claim 2, wherein
the integration device adds the signal delay amount to time information included in the image signal received by the reception device, and
the reception device outputs the received image signal at the timing determined by the time information and the signal delay amount.

4. The signal processing system according to claim 3, wherein the time information includes imaging time by the imaging device.

5. The signal processing system according to claim 1, wherein the integration device sets the signal delay amount on a basis of a transmission time from the transmission device to the processing device, a processing time in the processing device, and a transmission time from the processing device to the reception device.

6. The signal processing system according to claim 5, wherein the integration device calculates the processing time using a delay amount table regarding the signal processing by the processing device provided on the cloud.

7. The signal processing system according to claim 6, wherein
a plurality of the processing devices is provided on the cloud, and
the processing time includes a transmission time between the processing devices.

8. The signal processing system according to claim 1, wherein the processing device performs the signal processing on the image signal in a unit of a divided image obtained by dividing a frame.

9. The signal processing system according to claim 8, wherein in a case where the signal processing is performed on a plurality of the image signals, the processing device executes synchronization in a unit of the frame on a basis of a result of the signal processing performed in the unit of the divided image.

10. The signal processing system according to claim 9, wherein the integration device sets, as a method for the synchronization executed in the unit of the frame, either latency-prioritized synchronization in which a first image signal is not delayed but is synchronized with a second image signal preceding by a predetermined number of frames, or time-prioritized synchronization in which the first image signal is delayed and synchronized in accordance with the second image signal.

11. The signal processing system according to claim 10, wherein
the imaging device includes a medical device, and
the integration device sets either the latency-prioritized synchronization or the time-prioritized synchronization on a basis of a type of the medical device.

12. The signal processing system according to claim 10, wherein the processing device performs either the latency-prioritized synchronization or the time-prioritized synchronization using time information, a frame number, and a divided image number included in the image signal.

13. A signal processing method comprising
setting, by an integration device of a signal processing system, a signal delay amount from a transmission device to a reception device in accordance with a signal processing content of a processing device and transmitting the signal delay amount to the reception device,
the signal processing system including:
the transmission device that transmits an image signal from an imaging device;
the processing device that is provided on a cloud and performs signal processing on the transmitted image signal;
the reception device that receives the image signal subjected to the signal processing; and
the integration device that is connected to a network together with the transmission device, the processing device, and the reception device which are time-synchronized.
